(19) United States
(12) Patent Application Publication
(10) Pub. No.: US 2023/0097076 A1
(43) Pub. Date: Mar. 30, 2023

(54) PHARMACEUTICAL FORMULATION CONTAINING A PSYCHEDELIC SUBSTANCE OBTAINED BY SELECTIVE LASER SINTERING (SLS) 3D PRINTING

(71) Applicant: Biomind Labs UK Limited, Birmingham (GB)

(72) Inventor: Alejandro Carlos Antalich Raibar, Montevideo (UY)

(73) Assignee: Biomind Labs UK Limited, Birmingham (GB)

(21) Appl. No.: 17/956,528

(22) Filed: Sep. 29, 2022

Related U.S. Application Data
(60) Provisional application No. 63/293,971, filed on Dec. 27, 2021.

(30) Foreign Application Priority Data
Sep. 29, 2021 (UY) 100135
Dec. 30, 2021 (EP) 21218273.7

(51) Int. Cl. A61K 31/675; A61K 31/4045; A61K 31/137; A61K 9/24; A61K 9/00; A61K 9/20; B33Y 80/00; B33Y 10/00; B29C 64/153; B29C 64/245

(52) U.S. Cl. CPC A61K 31/675; A61K 31/4045; A61K 31/137; A61K 9/209; A61K 9/006; A61K 9/2095; B33Y 80/00; B33Y 10/00; B29C 64/153; B29C 64/245; B29L 2031/753

(57) ABSTRACT

The present invention discloses solid oromucosal pharmaceutical formulations containing a psychedelic selected from psilocybin, psilocin, or mescaline and/or an analog thereof as an active ingredient, a method of preparation of the pharmaceutical form by Selective Laser Sintering (SLS) 3D printing, and treatment of neurological and/or psychiatric disorders, as well as inflammatory disorders.

### CROSS-REFERENCE TO RELATED APPLICATIONS

The application claims priority to Uruguayan Patent Application No. UY 100135, filed on Sep. 29, 2021, U.S. Provisional Application No. 63/293,971, filed Dec. 27, 2021, and European Patent Application No. 21218273.7, field Dec. 30, 2021. The entire contents of each are hereby incorporated by reference in their entirety.

### FIELD OF THE INVENTION

The present invention refers to a solid pharmaceutical formulation containing a psychedelic molecule selected from psilocybin, psilocin, or mescaline and/or analogues thereof as the active ingredient for oromucosal delivery. The formulation is obtained by Selective Laser Sintering (SLS) 3D printing, and can be used in the treatment of neurological and/or psychiatric disorders, as well as various inflammatory disorders.

### BACKGROUND OF THE INVENTION

Psilocybin, psilocin, and mescaline are psychedelic psychotropic substances that cause perceptive changes when consumed.

Psilocybin is a natural compound that is found in variable concentrations in more than 200 species of basidiomycete fungi. (Berger K. J. and Guss D. A., The Journal of Emergency Medicine, 2005; 28(2):175-183: “Mycotoxins revisited: Part II;” Guzmán, G., Economic Botany, 2008, 62(3):404-412: “Hallucinogenic Mushrooms in Mexico: An Overview;” Burillo-Putze G, et al., Anales del Sistema Sanitario de Navarra, 2013, 36(3):505-518: “Drogas emergentes (III): plantas y hongos alucinógenos.”) Psilocybin (4-phosphoryl-4-hydroxy-N, N-dimethyltryptamine) and psilocin (4-hydroxy-N,N-dimethyltryptamine) are considered to be tryptamine derivatives (FIG. 1), due to their central indole ring structure. Psilocybin and psilocin are also very structurally similar to the neurotransmitter serotonin (5-hydroxytryptamine).

Mescaline (3,4,5-trimethoxyphenetylamine) is a phenylethylamine (FIG. 1), which is mainly found in the Peyote cactus (Lophophora williamsii). Structurally, it consists of three methoxide groups attached to a benzene ring in positions 3, 4, and 5, in addition to an aliphatic side chain with an amino group. It has a structural resemblance to the neurotransmitter dopamine (Kulma A. and Szopa J., Plant Science, 2007; 172(3):433-440: “Catecholamines are active compounds in plants.”)

Tryptamines and phenylethylamines effect the serotonergic and dopamine systems, and, depending on the compound, produce effects in other neurotransmission systems, such as the adrenergic system. These hallucinogens have clinical effects on various biological systems and functions of the body. (Fantegrossi W. E. et al., Pharmacology, biochemistry, and behavior, 2008, 88:358-365: “Hallucinogen-like effects of N,N-dipropyltryptamine (DPT): possible mediation by serotonin 5HT1A and 5-HT2A receptors in rodents;” McKenna D. J. et al., Neuropharmacology, 1990, 29:193-198: “Differential interactions of indolealkylamines with 5-ydroxytryptamine receptor subtypes; Rickli A. et al., European neuropsychopharmacology, 2016, 26:1327-1337: “Receptor interaction profiles of novel psychoactive tryptamines compared with classic hallucinogens.”) Scientific studies focused on their potential therapeutic uses evidence the benefits of using these compounds to treat various types of ailments and diseases. These molecules have been shown to have a well-established physiological and psychological safety profile in laboratory and clinical research and are non-addictive. (Johnson M., et al., Journal of Psychopharmacology, 2008, 22:603-620: “Human hallucinogen research: guidelines for safety.”)

Psilocybin, psilocin, and mescalin have been studied by scientists, psychiatrists, and mental health professionals since the 1960s, and these and other psychedelics have shown efficacy as a promising treatment alternative for a wide range of psychiatric disorders. In recent years, these molecules have been the subject of various preclinical and clinical trials. (Kyzar et al., Trends in Pharmacological Science, 2017, 38(11):992-1005: “Review Psychedelic Drugs in Biomedicine.”) Psilocybin is, to date, the most clinically researched psychedelic. Its potential for the treatment of health problems such as depression, obsessive-compulsive disorder (OCD), anxiety, and addiction to drugs, tobacco, and alcohol has been demonstrated. Further, there are studies related to eating disorders, specifically anorexia nervosa. (de Veen B. T. et al., Expert Review of Neurotherapeutics, 2016, 17:203-212: “Psilocybin for treating substance use disorders;” Ross S., Psychiatric Clinics of North America, 2012, 35(2):357-374: “Serotonergic hallucinogens and emerging targets for addiction pharmacotherapies;” Johnson M. W. et al., Journal of Psychopharmacology, 2012, 28(11):983-992: “Pilot study of the 5-HT2AR agonist psilocybin in the treatment of tobacco addiction;” Grob C. S. et al., Archives of General Psychiatry, 2011, 68:71-78: “Pilot-study of psilocybin treatment for anxiety in patients with advanced-stage cancer;” Griffiths R. R. et al., Journal of Psychopharmacology, 2016, 28:1181-1197: “Psilocybin produces substantial and sustained decreases in depression and anxiety in patients with life-threatening cancer: a randomized double-blind trial;” Ross S. et al., Journal of Psychopharmacology, 2016, 30:1165-1180: “Rapid and sustained symptom reduction following psilocybin treatment for anxiety and depression in patients with life threatening cancer: a randomized controlled trial;” Carhart-Harris R. L. et al., Lancet Psychiatry, 2016, 3:619-627: “Psilocybin with psychological support for treatment-resistant depression: an open-label feasibility study;” Winkelman M., Current drug abuse reviews, 2014, 72:10116: Psychedelics as medicines for substance abuse rehabilitation: evaluating treatments with LSD, Peyote, Ibogaine and Ayahuasca.”)

Another emerging therapeutic area that is very promising for psychedelics is their use as anti-inflammatory agents. Activation of 5-HT2A receptors produces potent anti-inflammatory effects in animal models of human inflammatory disorders at sub-psychedelic levels, i.e. at levels below which psychedelic effects are observed. In general, psychedelics regulate inflammatory pathways through novel mechanisms and represent a new and exciting treatment strategy for various inflammatory disorders. (Flanagan T. W. and Nichols C. D., International Review of Psychiatry, 2018, “Psychedelics as anti-inflammatory agents.”)

In 2018, the Food and Drug Administration (FDA) designated psilocybin therapy as a “Breakthrough Therapy” for treatment-resistant depressions, and in 2019, for major depressive disorder. Studies have shown that psilocybin is well tolerated and safe for human studies at oral doses of 8 to 25 mg and intravenous doses of 1 to 2 mg. (Passie T. et al., Addiction Biology, 2002, 7:357-364: “The pharmacology of psilocybin.”)

When psilocybin is administered orally or parenterally it dephosphorylates quickly and is almost completely transformed into psilocin. (¹ Horita A., Weber U., Biochemical Pharmacology, 1961, 7:47-54: “The enzymic dephosphorylation and oxidation of psilocybin and psilocin by mammalian tissue homogenates;” Hasler F. et al., Pharmaceutica acta Helvetiae, 1997, 72:175-184: “Determination of psilocin and 4-hydroxyindole-3-acetic acid in plasma by HPLC-ECD and pharmacokinetic profiles of oral and intravenous psilocybin in man.”) Therefore, psilocin is believed to be responsible for the psychoactive effects. However, when psilocybin is administered orally or parenterally, it is metabolized mainly in the liver, suffering an important first-pass effect, which considerably reduces its concentration before systemic circulation.

When administered intravenously, psilocybin is converted to psilocin in the kidneys, a process that may be less efficient. (Hasler F., et al., Journal of pharmaceutical and biomedical analysis, 2002, 30:331-339: “Renal excretion profiles of psilocin following oral administration of psilocybin: a controlled study in man.”)

Pharmacokinetically, significant amounts of psilocin are detectible in plasma within 20 to 40 minutes after oral administration of psilocybin, and maximum concentrations are reached after approximately 80 to 100 minutes. (Hasler F. et al., Pharmaceutica acta Helvetiae, 1997, 72:175-184: “Determination of psilocin and 4-hydroxyindole-3-acetic acid in plasma by HPLC-ECD and pharmacokinetic profiles of oral and intravenous psilocybin in man;” Lindenblatt H., et al., Journal of chromatography. B, Biomedical sciences and applications, 1998, 709:225-263: “Quantitation of psilocin in human plasma by high performance liquid chromatography and electrochemical detection: comparison of liquid-liquid extraction with automated on-line solid-phase extraction;” Passie T., et al., Addiction Biology, 2002, 7:357-364: “The pharmacology of psilocybin.”) Different studies show that psilocybin and psilocin have a plasma elimination half-life of approximately 50 to 160 minutes. (Hasler F., et al., Pharmaceutica acta Helvetiae, 1997, 72:175-184: “Determination of psilocin and 4-hydroxyindole-3-acetic acid in plasma by HPLC-ECD and pharmacokinetic profiles of oral and intravenous psilocybin in man;” Martin R, et al., International journal of legal medicine, 2013b, 127:593-601: “Determination of psilocin, bufotenine, LSD and its metabolites in serum, plasma and urine by SPE-LC-MS/MS;” Martin R., et al., International journal of legal medicine, 2012, 126:845-849: “A validated method for quantitation of psilocin in plasma by LC-MS/MS and study of stability.”) Typically, the effects of psilocin disappear completely between 4 and 6 hours after administration. (Shulgin A. T., Journal of psychedelic drugs, 1980, 12:79: “Profiles of psychedelic drugs. Psilocybin.”)

Mescaline is rapidly absorbed in the gastrointestinal tract and distributed to the kidneys and liver, where it combines with liver proteins, which delays its concentration in the blood. Its metabolization in the body occurs mainly in the liver by the action of an amino oxidase enzyme. (Kapadia G. J. and Fayez M. B., Journal of pharmaceutical sciences, 1970, 59(12):1699-1727: “Peyote constituents: chemistry, biogenesis, and biological effects;” Dasgupta A., Advances in clinical chemistry, 2017, 78:163-186: “Challenges in Laboratory Detection of Unusual Substance Abuse: Issues with Magic Mushroom, Peyote Cactus, Khat, and Solvent Abuse;” Hilliker K. S. and Roth R. A., Biochemical Pharmacology, 1980, 29(2):253-255: “Prediction of mescaline clearance by rabbit lung and liver from enzyme kinetic data.”) Orally, the minimum active dose of mescaline is around 100 milligrams. A dose of 500 or 600 milligrams produces a very intense visionary experience, which will last between 6 and 10 hours.

### SUMMARY

One general aspect of the invention discloses a solid pharmaceutical formulation for oromucosal delivery including i) a psychedelic compound selected from psilocybin, psilocin, mescaline, or an analog thereof; and ii) a thermoplastic polymer. The formulation may also include one or more excipients. In this embodiment, the solid formulation can be an orodispersible formulation. In this embodiment the solid formulation can be porous.

In an embodiment, the solid pharmaceutical formulation can include about 90-95 wt. % of the thermoplastic polymer, about 3-5 wt. % of one or more excipients, and about 3-5 wt. % of the psychedelic molecule. The thermoplastic polymer can include polyvinylpyrrolidone (PVP), hydroxy propyl methylcellulose (HPMC), polycaprolactone (PCL), poly-L-lactic acid (PLLA), polyethylene (PE), high density polyethylene (HDPE), polyethylene oxide (PEO), and ethyl cellulose (EC).

In an embodiment, the solid pharmaceutical formulation can have a hardness of 2-6 kg and a friability of less than 1.5.

In an embodiment, the solid pharmaceutical formulation can undergo complete dissolution in less than about 10 minutes.

In an embodiment the solid pharmaceutical formulation is a tablet.

In an embodiment, the solid pharmaceutical formulation is substantially free of water.

Another general aspect of the invention discloses a method of treating a neurological and/or psychiatric disorder and/or inflammatory disorder by administering to a patient in need thereof the solid pharmaceutical formulation to provide oromucosal delivery.

Another general aspect of the invention discloses a method of making the solid pharmaceutical formulation, the method including:
i. mixing the psychedelic molecule, thermoplastic polymer, and optionally one or more excipients together to form a mixture;
ii. dispensing the mixture into a dust bed; and
iii. sintering the dust to form the solid pharmaceutical formulation.

In this embodiment the thermoplastic polymer can include polyvinylpyrrolidone (PVP), hydroxy propyl methylcellulose (HPMC), polycaprolactone (PCL), poly-L-lactic acid (PLLA), polyethylene (PE), high density polyethylene (HDPE), polyethylene oxide (PEO), and ethyl cellulose (EC).

In this embodiment the dust bed is part of a 3D printer and sintering includes Selective Laser Sintering using the 3D printer. In this embodiment the step of distributing the mixture includes depositing the dust onto the dust bed and distributing the dust using a roller. In this embodiment the dust bed is a piston.

### BRIEF DESCRIPTION OF THE DRAWINGS

FIG. 1 shows the structures of psilocybin, psilocin, mescaline, tryptamine, serotonin, and dopamine.

FIG. 2 shows an administration scheme for a 3D-SLS printed oromucosal composition.

FIG. 3 shows a graphic scheme of the Selective Laser Sintering (SLS) process.

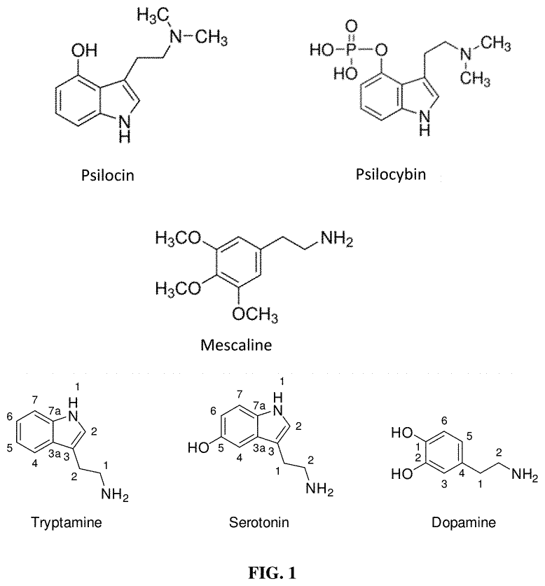

### DETAILED DESCRIPTION OF THE INVENTION

Disclosed herein are solid pharmaceutical formulations for oromucosal delivery obtained by 3D printing by Selective Laser Sintering (SLS) containing a psychedelic molecule selected from psilocybin, psilocin and/or mescaline and/or analogues thereof as the active ingredient. As shown in FIG. 3, the SLS 3D printing system uses a Laser (1) coupled to a scanning system (2), and a dust dispensing system (3) that distributes the reactive dust which includes the drug and a thermoplastic polymer. The distributions system expels the dust onto the surface of a piston which forms a manufacturing platform (4) with the dust evenly distributed on the surface using a roller (5). The piston (6) lowers the manufacturing platform (7), which can hold a solid pharmaceutical form or mold (8) during the printing process. FIG. 3 also shows the Laser scanning direction (A), Sintered dust particles (B), Laser beam (C), Laser sintering of dust particles (D), dust particles pre-positioned for sintering (E), and the pharmaceutical mold or form (F).

Pharmaceutical formulations include tablets.

Unless otherwise indicated, all parts and percentages are by weight. As used herein, the term “about” refers to plus or minus 10% of the indicated value. Unless otherwise stated or made clear by context, weight percentages are provided based on the total amount of the composition in which they are described. As used herein, the singular forms “a,” “an,” and “the” include plural referents unless the context clearly dictates otherwise.

Oral administration of drugs is preferred by patients for comfort and safety. Up to 70% of adults prefer solid, oral dosage forms over alternative administration routes, and it has been shown that oral dosage forms increase patient compliance due to ease of handling. Even the packaging of oral dosage forms increases compliance. For example, it is simpler for a patient to retrieve a tablet dosage from a blister package as compared to other, alternative formulations, such as films, solutions, or powders. However, for some drugs, oral administration has a series of disadvantages, including exposure of the active drug to the aggressive gastrointestinal environment which degrades bioactive molecules and limits absorption. This negatively impacts bioavailability and pharmacotherapy. Some of these disadvantages can be partially overcome through parenteral administration, although it is not the preferred choice of patients due to its invasiveness. (Dubey, S. K. et al., Drug Discovery Today, 2021, 26(4):931-950: “Oral peptide delivery: challenges and the way ahead.”)

Transmucosal oral (or oromucosal) administration (use of the mucosa available in the oral cavity) of drugs is a very promising alternative to the oral and parenteral pathways, and avoids disadvantages of oral administration while providing several additional advantages. For example, oromucosal delivery reduces first-pass metabolism and thus prevents gastrointestinal degradation. Oromucosal delivery also provides a direct route to systemic and central nervous system administration resulting in a rapid onset of action of the target drug. It is also a practical and painless route of application, and its use is not yet as widespread in the pharmaceutical industry as traditional routes of administration.

In the design of a transmucosal oral formulations there are important aspects to consider, including the need for rapid decay of the formulation, and the subsequent dissolution of the drug in a reduced volume of fluid (saliva). These issues are addressed by using compression technology or molding solid formulations with high porosity and utilizing highly effective disintegrants. However, these formulations strategies do not allow precise dosing adjustments based on the needs of patients. (Mathias N. R. et al., Journal of Pharmaceutical Sciences, 2010, 99(1):1-20: “Non-invasive Systemic Drug Delivery: Developability Considerations for Alternate Routes of Administration.”)

Oromucosal delivery systems allow for administration of active ingredients with easy absorption by the body, and reduces or avoids first pass metabolism in the liver, and therefore achieves better bioavailability. Oromucosal delivery systems release the active ingredient in the oral cavity and allow for absorption of the active ingredient through the buccal mucosa. Oromucosal delivery systems can also be orodispersible. As used in the context of the present disclosure, orodispersible means the delivery system/formulation fully disintegrates in the oral cavity/mouth These systems successfully avoid first pass metabolism, which is also known as the first pass effect.

The first pass effect is a phenomenon in which a drug gets metabolized at a specific location in the body that results in a reduced concentration of the active drug upon reaching its site of action or systemic circulation. The first pass effect is often associated with the liver, which is the major site of drug metabolism. However, the first pass effect can also occur in the lungs, vasculature, gastrointestinal tract, and other metabolically active tissues in the body. The effect can also be augmented by various factors, including plasma protein concentrations, enzymatic activity, and gastrointestinal motility.

Drug delivery via the oral mucous membranes is an attractive, alternative to other oral administration methods in which the active ends up in the gastrointestinal tract and offers several advantages over other methods. In particular, oromusocal administration delivers the active drug via the sublingual mucosa (i.e., the membrane of the ventral surface of the tongue and the floor of the mouth) where it is readily absorbed and enters into systemic circulation while bypassing the gastrointestinal tract. This avoids first pass effects and results in improved bioavailability, rapid onset of drug action, and improved patient compliance, as well as complete avoidance of dysphagia (i.e., difficulty in swallowing).

Use of 3D printing technology is a novel and emerging application within the pharmaceutical industry that has the potential to solve issues commonly associated with preparation of a transmucosal formulation.

Additive manufacturing by 3D printing is a layer-by-layer manufacturing process that allows three-dimensional pharmaceutical forms to be produced from digital designs. There are numerous processes adaptable to additive manufacturing, with Selective Laser Sintering (SLS) being one of the most promising. (Kulinowski, P. et al., Additive Manufacturing, 2021, 38:101761: “Selective laser sintering (SLS) technique for pharmaceutical applications Development of high dose controlled release printlets.”)

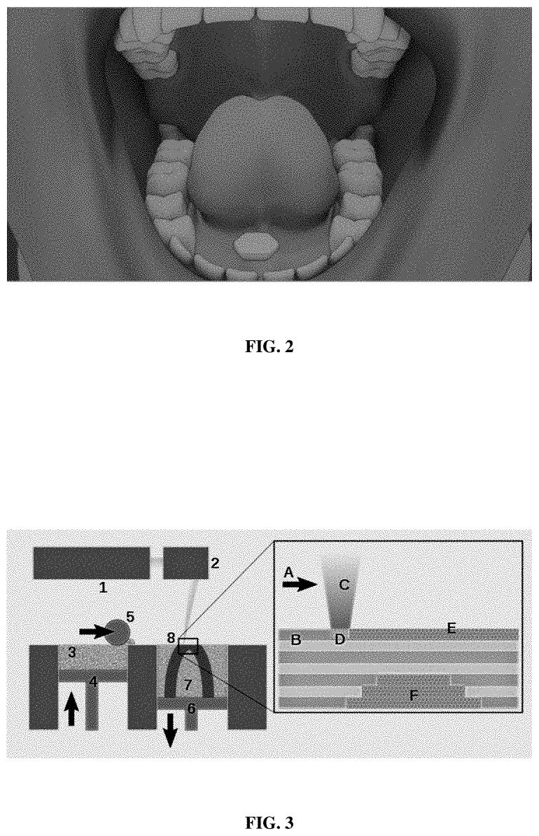

The SLS technique can be used to obtain highly porous pharmaceutical forms with very low decay times, which is ideal for oromucosal administration via the sublingual route (FIG. 2). Using this technique results in a rapid onset of action and a low hepatic first pass effect. Additionally, printing software can be modified to obtain high-dose formulations and different controlled-release particles can be printed to modify and adapt the dosage in real time for personalization to a particular subject.

Macro- and micro-structure conformations can be printed to adjust the dissolution profile of the active ingredients and allows for the design of flexible dosage forms for personalized medicine. (Awad A. et al., International Journal of Pharmaceutics, 2020, 586:119594: “3D printing: Principles and pharmaceutical applications of selective laser sintering;” Fina, F. et al., International Journal of Pharmaceutics, 2018, 547(1-2):44-52: “3D printing of drug-loaded gyroid lattices using selective laser sintering;” Thakkar R. et al., European Journal of Pharmaceutics and Biopharmaceutics, 2021, 163:141-156: “Synergistic application of twin-screw granulation and selective laser sintering 3D printing for the development of pharmaceutical dosage forms with enhanced dissolution rates and physical properties.”)

U.S. Pat. No. 5,490,962 describes the preparation of dosage forms using Solid Freeform Fabrication (SFF) methods. These methods can be adapted to be used with a variety of different materials to create dosage forms with defined compositions, forces, and densities. SLS is an example of a SFF method and allows for manipulation of the macrostructure and porosity of the dosage forms by manipulating the 3D printing parameters, the type of polymer, and the size of the particles, as well as the solvent and/or ligand. WO98/36739 references U.S. Pat. No. 5,490,962, emphasizing that in certain therapies, a pulsed release of drugs for long periods of time is required. Therein a multiphase dosing method is described, which provides a differential release of the different drugs through 3D printing.

US 2019/0374471 and US 2021/0169811 discuss the use of 3D printing by SLS to obtain a wide variety of formulations (capsules, films, patches) pre-loaded with a drug to achieve a wide range of dissolution averages and decay speeds, depending on the composition of the formulations. U.S. Pat. No. 10,350,822 and WO 2020/148442 disclose oral dosing forms composed of multiple layers of a material mixed with an active ingredient of different chemical nature that are obtained by 3D printing. These types of technology potentially allow for the adjustment of drug release profiles according to the needs of the therapy.

Conventional pharmaceutical formulation technology does not allow for modification of dosage formulations in real time. Complex changes to manufacturing equipment are necessary to change simple characteristics, such as geometry, dosage, and color, among others.

Compared with conventional formulation techniques, SLS 3D printing has competitive advantages that improve the safety, effectiveness, and accessibility of medicines by allowing for the personalized selection of formulation components, such as excipients, design, dosage, and color.

This technology also allows for the development of complex products, products on demand and/or personalized products. For example, geometry (shape) and/or color can be readily changed in SLS 3D printing to increase adherence to dosing regimens for marketing and treatment purposes.

In the development and production of personalized medicines, SLS 3D printing allows for the proper selection of manufacturing parameters. For example, tablets can be produced with the exact dose of the active ingredient that suits the individual needs of each patient. SLS 3D printing also allows for the design of a pharmaceutical formulation with controlled time release of the drug it contains and optimize its onset of action Geometries can also be varied (for example by changing thickness, layering and surface area) or components selected to alter release characteristics and other properties of the formulations. Release characteristics can include, for example, the rate of release of the active, the duration of release, and the total amount of active delivered.

The SLS 3D printing process consists of a laser that sweeps the surface of a bed of powder to sinter or melt it. The bed of dust is then displaced downwards, new dust is distributed above it, and the process is repeated to produce a solid object.

Sintering generally occurs at relatively high temperatures, limiting its applicability to active ingredient that can survive the process without degradation. Psilocybin, psilocin, and mescaline can withstand the high sintering temperatures by selecting the appropriate polymers.

While there are pharmaceutical compositions known in the art containing at least one of the psychedelic molecules psilocybin, psilocin and/or mescaline, and strategies to avoid first pass metabolism of the psychedelic, there is no disclosure that proposes a formulation suitable for oromocusal administration. The present invention provides a novel solution, using SLS 3D printing to manufacture the oromucosal formulation.

Solid oromucosal pharmaceutical formulations are disclosed containing any of the following psychedelic molecules: psilocybin, psilocin, mescaline, and/or pharmaceutically acceptable salts or analogues thereof, as an active ingredient, and the formulations are used in the treatment of neurological and/or psychiatric disorders. Alternative psychedelics for use in the solid oromucosal pharmceuticals formulations include, but are not limited to, DMT, 5-MeO-DMT, and pharmaceutically acceptable salts and/or analogs thereof. The formulations can also be used as anti-inflammatory agents for treating various inflammatory disorders.

Use of 3D SLS printing of formulations provides opportunities to vary properties to achieve advantageous formulations. For example, the formulations can be manufactured to have a desired porosity to allow dispersion of the actives. Porosity, as used in the context of the present disclosure, is the ratio of the total volume of the voids between particles and the pores of the formulation, to the total volume of the powder or tablet including its voids and pores. The formulations can also be manufactured to have high-water capture. The high-water capture is achieved by including hydrophilic polymers and/or excipients in the formulation. These hydrophilic polymers or excipients quickly capture water from the saliva to promote rapid dissolution of the formulation for oromucosal delivery.

The orodispersible tablets of the present invention first disintegrate into fragments within the oral cavity/mouth of the patient exposing the psychedelic compound. The psychedelic compound are then dissolved (i.e., undergo dissolution) by the saliva in the oral cavity/mouth, resulting in oromucosal delivery of the psychedelic to the patient.

In exemplary embodiments, the material is sintered at a temperature of between about 90 and about 130° C., and the material contains about 3-5% of the psychedelic active ingredient, about 90-95% of thermoplastic polymer, and about 0-5%, for example about 3-5% of additional excipients. A solid pharmaceutical formulation is obtained with mechanical properties suitable for packaging and administration. For example, the formulation can have a hardness of about 2-6 kg and a friability of less than about 1.5. The orodispersible tablets disintegrate in the oral cavity and the psychedelic compound undergoes complete dissolution in less than about 10 minutes, less than about 9 minutes, less than about 8 minutes, less than about 7 minutes, less than about 6 minutes, less than about 5 minutes, less than about 4 minutes, less than about 3 minutes, less than about 2 minutes, less than about 1 minute, or less than about 30 seconds.

Suitable thermoplastic polymers for use in the formulations include, but are not limited to, polyvinylpyrrolidone (PVP), hydroxy propyl methylcellulose (HPMC), polycaprolactone (PCL), poly-L-lactic acid (PLLA), polyethylene (PE), high density polyethylene (HDPE), polyethylene oxide (PEO), and ethyl cellulose (EC), among others. In exemplary embodiments, the thermoplastic polymer is hydrophilic. Other hydrophilic excipients, such as binders and dispersants, are known in the art and can be suitably selected by the skilled artisan to provide the desired solubility and water capture properties. Exemplary excipients include, but are not limited to, cross-linked cellulose, starch, and starch derivatives.

Water is known to contaminate and degrade hydrophilic thermoplastic polymers. Therefore, in order to increase and/or maintain the chemical and microbiological stability of the formulations of the present invention, the formulations are substantially free of water. Substantially free of water is defined as less than about 5 wt. %, about 4 wt. %, about 3 wt. %, about 2 wt. %, about 1 wt. %, about 0.5 wt. %, or about 0.1 wt. % of water based on the total weight of the formulation. Preferably the formulation contains less than about 5 wt. % water. Most preferably the formulation does not contain any added water.

The invention is exemplified in more detail in the two non-limiting examples below.

### Example 1

A psychedelic molecule (psilocybin, psilocin or mescaline and/or analogues thereof), a thermoplastic polymer and, optionally, other the excipients are passed through a 100-mesh sieve and ground if necessary. Then all the ingredients are mixed for about 15 minutes. They are then inserted into the dust bed of the 3D printer, with a distance of about 1.0 mm between the laser and powder bed. Then, the printing platform is moved along the Z axis. Excess dust is removed using a device situated along the X axis. Sintering is accomplished by Laser printing using a printer equipped with a 450 nm laser generator.

The prints are designed using specific software and the laser power is set to about 0.05-3.5 W with a print depth (i.e., sintering depth) of between about 3%-80%.

The thickness is measured in the center or at the edge using a micrometer and then mechanical tests for hardness and friability are performed.

### Example 2

The psychedelic molecules (psilocybin, psilocin, or mescaline and/or analogues thereof) are mixed together with the inactive materials (polymers and excipients) in a double cone mixer for about 20 minutes.

The forms are designed with Autodesk Inventor 2020 and saved in the STL file format. Printing is done using a 2.3 W blue laser printer (445 nm wavelength).

The temperature of the internal printing camera and the temperature of the dust surface is experimentally adjusted, and the laser scanning speed is set at 100 mm/s. The thickness of the layer of dust is set to 100 μm.

Compact prints are obtained at an internal chamber temperature of about 140° C. and a dust surface temperature of about 155° C.

The thickness is measured in the center or at the edge using a micrometer and then mechanical tests for hardness and friability are performed.

The invention is described herein by the non-limiting examples intended only to teach those skilled in the art the best way known to the inventors to make and use the invention. Nothing in the examples or elsewhere in the specification should be considered as limiting the scope of the present invention. The specific embodiments of the invention described may be modified or varied, without departing from the invention, as appreciated by those skilled in the art in light of the above teachings. It is therefore to be understood that, within the scope of the claims and their equivalents, the invention may be practiced otherwise than as specifically described.

1. A solid pharmaceutical formulation for oromucosal delivery comprising i) a psychedelic compound selected from the group consisting of psilocybin, psilocin, mescaline, or an analog thereof; and ii) a thermoplastic polymer.

2. The solid pharmaceutical formulation of claim 1, further comprising one or more excipients.

3. The solid pharmaceutical formulation of claim 1, wherein the formulation is orodispersible.

4. The solid pharmaceutical formulation of claim 1, wherein the formulation is porous.

5. The solid pharmaceutical formulation of claim 1, wherein the formulation comprises about 90-95% of the thermoplastic polymer.

6. The solid pharmaceutical formulation of claim 1, wherein the formulation comprises about 3-5% of the psychedelic molecule.

7. The solid pharmaceutical formulation of claim 2, comprising about 3-5% of the psychedelic molecule, about 90-95% of the thermoplastic polymer, and about 3-5% of the one or more excipients.

8. The solid pharmaceutical formulation of claim 1, wherein the formulation has a hardness of about 2-6 Kg and a friability of less than about 1.5.

9. The solid pharmaceutical formulation according to claim 1, wherein the formulation undergoes complete dissolution in less than about 10 minutes.

10. The solid pharmaceutical formulation according to claim 1, wherein the thermoplastic polymer is selected from the group consisting of polyvinylpyrrolidone (PVP), hydroxy propyl methylcellulose (HPMC), polycaprolactone (PCL), poly-L-lactic acid (PLLA), polyethylene (PE), high density polyethylene (HDPE), polyethylene oxide (PEO), and ethyl cellulose (EC).

11. The solid pharmaceutical formulation according to claim 1, wherein the formulation is a tablet.

12. The solid pharmaceutical formulation according to claim 1, wherein the formulation is substantially free of water.

13. A method of treating a neurological and/or psychiatric disorder and/or inflammatory disorder comprising administering to a patient in need thereof the solid pharmaceutical formulation of claim 1 to provide oromucosal delivery.

14. A method of making the solid pharmaceutical formulation of claim 1, the method comprising:
i) mixing the psychedelic molecule, thermoplastic polymer, and optionally one or more excipients to form a mixture;
ii) dispensing the mixture into a dust bed; and
iii) sintering the dust to form the solid pharmaceutical formulation.

15. The method of claim 14, wherein the thermoplastic polymer is selected from the group consisting of polyvinylpyrrolidone (PVP), hydroxy propyl methylcellulose (HPMC), polycaprolactone (PCL), poly-L-lactic acid (PLLA), polyethylene (PE), high density polyethylene (HDPE), polyethylene oxide (PEO), and ethyl cellulose (EC).

16. The method of claim 14, wherein the dust bed is part of a 3D printer and sintering comprises Selective Laser Sintering using the 3D printer.

17. The method of claim 14, wherein the step of distributing the mixture comprises depositing the dust onto the dust bed and distributing the dust using a roller.

18. The method of claim 14, wherein the dust bed is a piston.

19. The method of claim 18, further comprising lowering the piston after the sintering step, dispensing a second layer of the mixture onto the solid pharmaceutical composition, and sintering the second layer of the mixture.

20. The method of claim 14, wherein the dust bed is a manufacturing platform and the manufacturing platform further comprises an pharmaceutical mold.
